(19) United States
(12) Patent Application Publication
(10) Pub. No.: US 2013/0289391 A1
(43) Pub. Date: Oct. 31, 2013

(54) System and Method Using Forward Looking Imaging for Valve Therapies

(71) Applicant: VOLCANO CORPORATION, San Diego, CA (US)

(72) Inventors: Oren Levy, Emerald Hills, CA (US); Byong-Ho Park, Cincinnati, OH (US); Russell W. Bowden, Tyngsboro, MA (US); Dietrich Ho, Mountain View, CA (US); Stan Thomas, Palo Alto, CA (US)

(73) Assignee: Volcano Corporation, San Diego, CA (US)

(21) Appl. No.: 13/871,533

(22) Filed: Apr. 26, 2013

Related U.S. Application Data
(60) Provisional application No. 61/639,672, filed on Apr. 27, 2012.

(51) Int. Cl. A61B 8/08; A61B 8/12; A61B 8/00; A61B 8/06

(52) U.S. Cl. CPC A61B 8/0833; A61B 8/0891; A61B 8/06; A61B 8/12; A61B 8/461; A61B 8/085
USPC 600/424; 600/443

(57) ABSTRACT

A system is provided for aortic valve imaging utilizing forward looking imaging sensors. A method of imaging the aortic valve is provided that can be utilized for diagnostic evaluation and the delivery of a therapy. In one form, the imaging system can be used to place a replacement aortic valve. In another aspect, an imaging system is combined with a valve replacement delivery system.

### CROSS-REFERENCE TO RELATED APPLICATIONS

The present application claims priority to and the benefit of U.S. Provisional Patent Application No. 61/639,672, filed Apr. 27, 2012, which is hereby incorporated by reference in its entirety.

### BACKGROUND

The present disclosure relates to methods and systems for evaluating and treating cardiac valves utilizing forward looking imaging devices.

An implantable valve, designated hereafter as a “prosthetic valve”, permits the repair of a valvular defect by a less invasive technique in place of the usual surgical valve implantation which, in the case of valvular heart diseases, requires thoracotomy and extracorporeal circulation. A particular use for a prosthetic valve concerns patients who cannot be operated on because of an associated disease or because of very old age or also patients who could be operated on but only at a very high risk.

Although prosthetic valves and the process for implanting them can be used in various heart valve diseases, the primary indication typically involves the aortic orifice in aortic stenosis, more particularly in its degenerative form in elderly patients. Aortic stenosis is a disease of the aortic valve in the left ventricle of the heart. The aortic valvular orifice is normally capable of opening during systole up to 2 to 6 cm, therefore allowing free ejection of the ventricular blood volume into the aorta. This aortic valvular orifice can become tightly stenosed, and therefore the blood cannot anymore be freely ejected from the left ventricle. In fact, only a reduced amount of blood can be ejected by the left ventricle which has to markedly increase the intra-cavitary pressure to force the stenosed aortic orifice to open. In such aortic diseases, the patients can have syncope, chest pain, and mainly difficulty in breathing. The evolution of such a disease is disastrous when symptoms of cardiac failure appear, since 50% of the patients die in the year following the first symptoms of the disease.

Minimally invasive techniques are known to provide some relief for this condition. For example, highly calcified valves may be treated in an attempt to remove the calcification and restore flexibility to the valve leaflets. Such a system and technique is described in U.S. Pat. No. 7,803,168 hereby incorporated by reference herein in its entirety. In addition to treatment options, a number of systems are available to minimally invasively place a prosthetic valve to replace the function of the diseased valve. Such systems and methods for implantation are disclosed in U.S. Pat. Nos. 7,101,396, 7,846,203, 7,892,281 and 7,914,569 each hereby incorporated by reference herein in their entirety.

While existing systems offer options for treatment, placement of the devices requires cumbersome internal imaging devices such as transesophageal echo systems and/or external imaging systems requiring radiation and large amounts of contrast media. The use of cumbersome internal imaging requires additional specialists, raises the cost of the procedure, general anesthesia, and patient discomfort post procedure. Similarly, the use of external imaging techniques with contrast media can be harmful to patients who are often already in a fragile condition due to underlying health issues associated with the advanced age of the patients that tend to be candidates for valve treatment therapies.

As a result, there is a need for improvements in the imaging systems that can be used to assist in valve evaluation, treatment and valve prosthesis placement.

### SUMMARY

In one aspect, the present disclosure provides a medical method comprising, positioning a guidewire and a forward looking imaging device in the vasculature of a patient and advancing the guidewire and imaging device to a valve. The method includes imaging the valve with the forward looking imaging device to obtain a valve image and crossing the valve with at least a distal portion of the guidewire utilizing the valve image.

In another aspect, the present disclosure provides a method of imaging the valve of the heart or an artificial heart valve. The method comprises positioning a forward looking imaging device in the vasculature of a patient, advancing the forward looking imaging device to the superior vena cava and/or right atrium of the heart, and aligning the forward looking imaging device to image the aortic valve of the heart or an artificial heart valve.

In still a further aspect, the present disclosure provides an imaging system having a processor configured to receive imaging signals from an aortic imaging device, a visual display, a forward looking imaging device sized for placement in the human aorta, the imaging device generating image signals, and a connection between the imaging device and the processor, the connection providing the image signals to the processor.

In still a further aspect, the present disclosure provides a combination imaging catheter and prosthetic valve delivery system. The imaging catheter may be positioned in a deliver device adjacent the prosthetic valve and advanced to the implantation site as a unit.

In yet a further aspect, the present disclosure provides a combination imaging catheter and contrast media delivery system. The system is configured to allow the imaging system to provide forward looking images to the user and also allows the user to deploy contrast media to the distal portion of the system.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIG. 1a is a partial view of the circulatory system of a patient.

FIGS. 1b-1d are enlarged partial cross sectional views of a heart.

FIG. 2a is perspective view of an imaging device according to one aspect of the invention.

FIG. 2b is a schematic of a coordinate system illustrating parallel and perpendicular deflections of an imaging device relative to an imaging plane.

FIG. 3 is a partial cross sectional view of the heart showing positioning of an imaging device according to another aspect of the invention.

FIGS. 4a and 4b are stylized views of an implant being positioned across the aortic valve.

FIG. 5 is a partial cross sectional view of the heart showing positioning of an imaging device according to another aspect of the invention.

FIGS. 6a and 6b are stylized views of imaging of the aortic valve from the superior vena cava.

FIGS. 7a-7d are in vivo images generated by the imaging device according to one aspect of the invention.

FIG. 8 is a stylized view of the aortic valve showing different fields of view obtained from the positions shown in FIGS. 6a and 6b.

FIGS. 9a and 9b are stylized views of the aortic valve in a closed position and an open position.

FIG. 10 is side plan view in partial cross section illustrating a valve prosthesis delivery system including a forward looking imaging device.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications in the described devices, instruments, methods, and any further application of the principles of the disclosure as described herein are contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

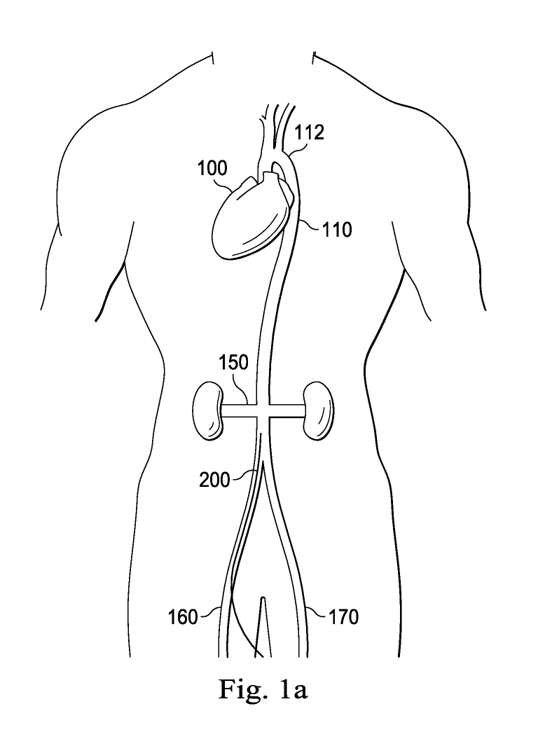

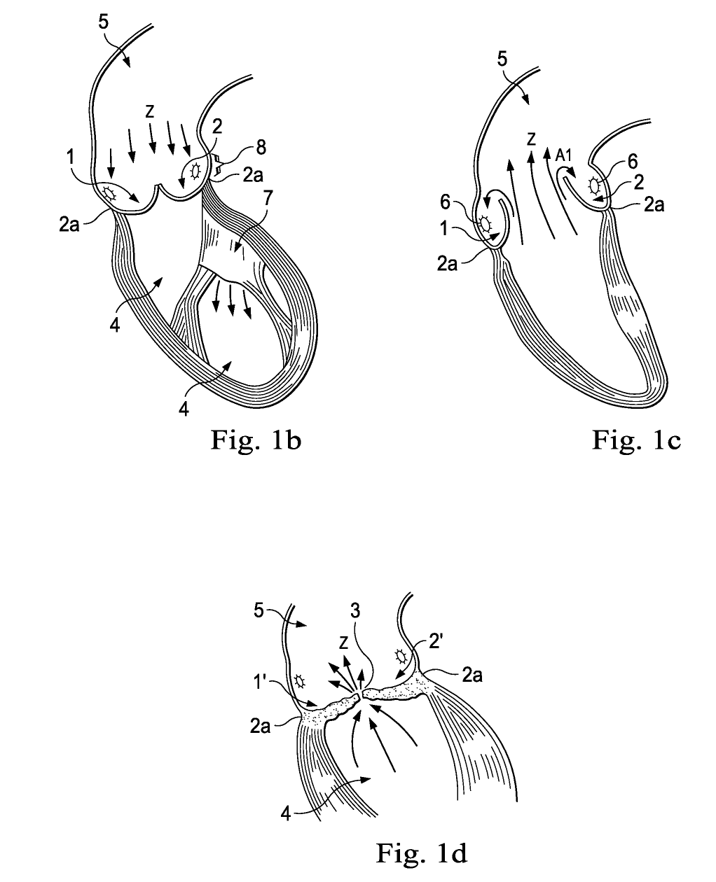

FIG. 1 illustrates a stylized representation of the vasculature of a patient. Beginning at the heart 100, blood is pumped into the aorta 110 through the aortic arch 112 as it exits the heart. The renal arteries 150 branch to the kidneys after which the aorta bifurcates to eventually form the femoral arteries 160 and 170. As will be described in greater detail below, a catheter 200 may be inserted into the femoral artery 160 and advanced along the aorta to gain access to the heart 100.

FIGS. 1b and 1c illustrate section views of a portion of the heart in the diastole and systole periods of the heart beat, respectively. The arrows Z indicate the general direction of the blood flow. The semi-lunar leaflets 1 and 2 of a native aortic valve (with only two out of three shown here) are thin, supple and move easily from the completely open position (systole) to the closed position (diastole). The leaflets originate from an aortic annulus 2a.

The leaflets 1′ and 2′ of a stenosed aortic valve as illustrated in FIG. 1d, are thickened, distorted, calcified and more or less fused, leaving only a small hole or a narrow slit 3, which makes the ejection of blood from the left ventricle cavity 4 into the aorta 5 difficult and limited. FIGS. 1b to 1d also show the coronary artery ostium 6 and FIG. 1b shows, in particular, the mitral valve 7 of the left ventricle cavity 4.

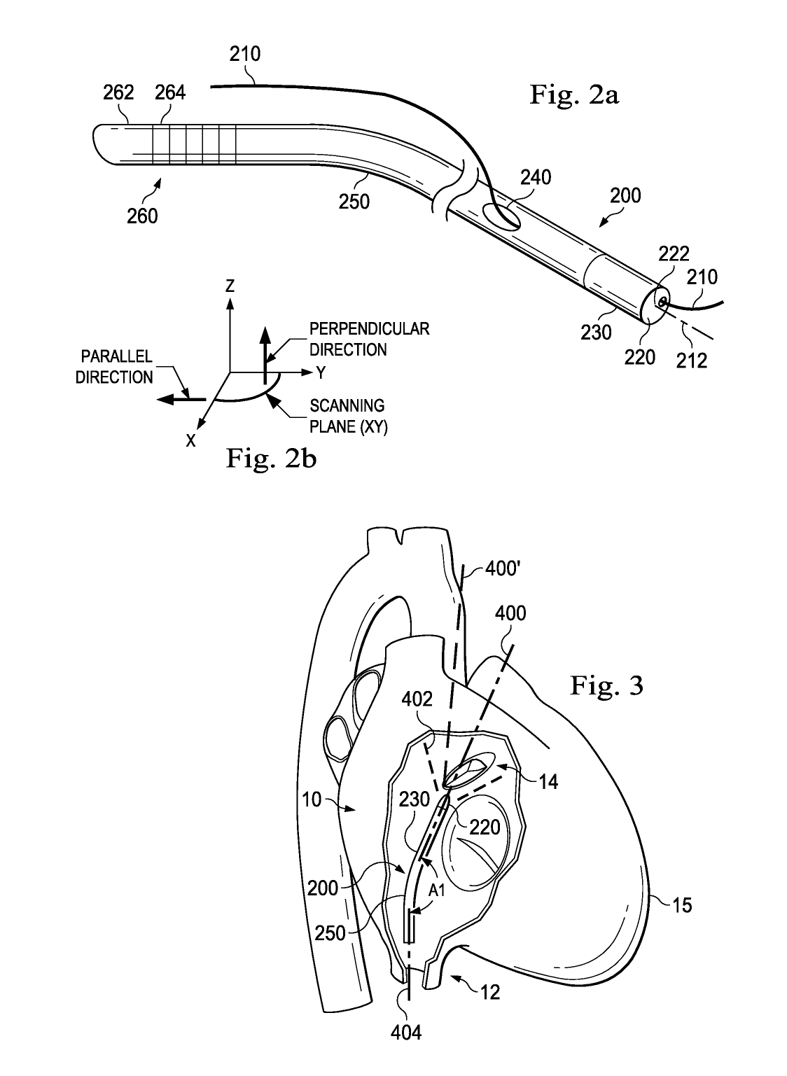

Referring to FIG. 2, shown therein are aspects of an imaging device 200 of an imaging system according to an embodiment of the present. More specifically, FIG. 2 is a diagrammatic schematic view of a portion of imaging device 200 that can be utilized to image portions of the heart and other portions of the vasculature. In the illustrated embodiment, the imaging device 200 is slidably positioned over a guidewire 210 over at least a portion of its distal end segment and exits the imaging catheter at side port 240. The guidewire exit port may also be at the very proximal end of the catheter. The imaging device 200 has a flexible elongate body 250 extending between the distal portion 230 and proximal portion 260. Distal portion 230 includes an imaging transducer for ultrasound imaging. Distal portion 230 can be constructed as disclosed in U.S. patent application Ser. No. 12/877,560, filed Sep. 8, 2010, titled “Devices, Systems and Methods for Field of View Control Imaging Systems” which is hereby incorporated by reference in its entirety

In some embodiments the elongate member 200 takes the form of a guidewire or catheter. In some instances, the imaging system as a whole, as well as the elongate member 200, and/or other aspects of the imaging system are similar to those described in U.S. Pat. No. 5,379,772, titled “FLEXIBLE ELONGATE DEVICE HAVING FORWARD LOOKING ULTRASONIC IMAGING,” U.S. Pat. No. 7,115,092, titled “TUBULAR COMPLIANT MECHANISMS FOR ULTRASONIC IMAGING SYSTEMS AND INTRAVASCULAR INTERVENTIONAL DEVICES,” and/or U.S. Pat. No. 7,658,715, titled “MINIATURE ACTUATOR MECHANISM FOR INTRAVASCULAR IMAGING,” each of which is hereby incorporated by reference in its entirety.

Proximal portion 260 includes a series of conducting rings 262 and 264 electrically coupled to conducting members extending within imaging device 200 to distal portion 230. Electrical conductors provide control, power and communications between the sensor assembly in distal portion 230 and a patient interface module (not shown). The electrical interface may be a connector positioned at the proximal end of the catheter or a pig tail cable extension. In one embodiment particularly suited for intracardiac echocardiography (ICE), device 200 has a 0.035 inch lumen to receive guidewire 210 and has a maximum outer diameter adjacent the tip 220 of between 10.5 French and 5.5 French. The guidewire lumen 240 is offset from the longitudinal axis 212 of the distal portion 230 containing the forward looking imaging system. The guidewire exiting on the distal end is visible within the planar imaging plane while not complete obscuring the ultrasound image. This is because the guidewire grazes the image plane cone without completely obscuring it. The guidewire assembly may also include a guidewire lumen that is sufficiently large to also deliver contrast injection when the guidewire is not present. This is useful when attempting to locate a particular lumen (i.e. CS ostium, LAA ostium, true lumen in an AAA case, etc.). The imaging area or scanning area of the imaging catheter may be isolated from the guidewire lumen such that fluids and blood are kept out of it. The device may also be deflectable at the distal end of the catheter by the use of pull wires or other deflection mechanisms. The deflection may be parallel or perpendicular to the image plane. A perpendicular deflection is useful in searching 3D space, while parallel deflection can help center an anatomical structure that may be at the far edge of the imaging plane. See FIG. 2b that illustrates the difference between parallel and perpendicular deflections. The deflection may range from ±5 degrees to ±90 degrees.

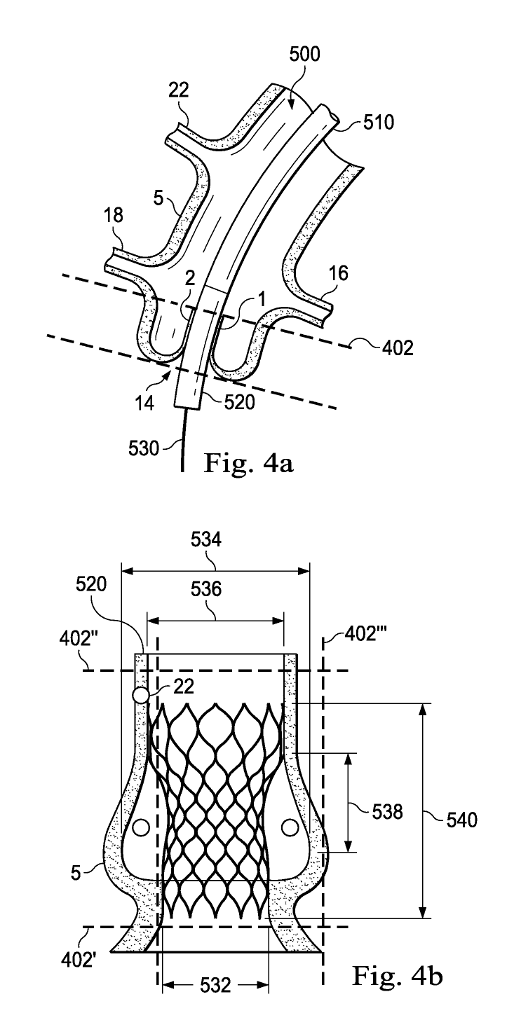

Referring now to FIG. 3, there is illustrated a heart 15 shown in partial cross section with a forward looking intra cardiac echocardiography (Forward Looking ICE) imaging device 200 disposed in the right atrium 10, made partially transparent in FIG. 4 so the internal structures may be illustrated. The catheter 200 is delivered into the right atrium from the inferior vena cava 12, which may be accessed in any known approach, such as for example, percutaneous access through the femoral vein. The major portion of the elongate flexible body 250 of the catheter remains in the inferior vena cava 12 while the distal portion 230 is deflected to extend along distal longitudinal axis 400. The distal portion 230 extends along longitudinal axis 400 at an angle A1 with respect to the longitudinal axis 404 of the elongate body. In the illustrated embodiment, the angle A1 is greater than 90°. Although angle A1 is illustrated as greater than 90°, differing patient anatomy may require alternative angulations. In the specific example illustrated, angle A1 is approximately 130°. Thus, obtaining the illustrated positioning in the right atrium the distal portion 230 must only to be deflected less than 50° from the angle of insertion along axis 404 to begin obtaining images of the aortic valve 14.

During use, once the distal tip 220 is positioned in the right atrium the user manipulates the tip 220 until an image of at least a portion of the aortic valve 14 is displayed. As shown in FIG. 3, the distal portion 230 is aligned with axis 400 which extends through at least a portion of the aortic valve. As will be understood, the distal tip 220 contains the forward looking ultrasound sensor which is generally centered along longitudinal axis 400. As a result, the ultrasound sensor of the distal tip 220 creates a field of view 402 that includes a substantial portion or complete portion of the aortic valve 14.

In one configuration of the forward looking ultrasound sensing system, the ultrasonic beam leaving the transducer has an approximate thickness of 1.5 mm which converges over approximately the first 6 mm and then diverges as it extends further from the transducer. The forward looking sensor can be constructed to provide a field of view of up to 180° although a typical system will utilize a 120° field of view. In one embodiment of the present invention, the field of view has been limited to approximately 60° to provide more detail of the aortic valve 14. The ultrasound beam can provide return imaging information for a tissue depth of approximately 5-7 cm depending upon the nature of the tissue being imaged. The scanned ultrasonic beam creates a fan shaped section of image data.

It will be appreciated that in one approach, the user positions the distal portion 230 to allow the forward looking sensor to image the aortic valve 14. When positioned along axis 400, the field of view 402 provides an image of the aortic valve 14 similar to that shown in FIG. 4a bracketed by the dashed lines presenting the anticipated field of view 402. The field of view of the forward looking system can be adjusted by moving the sensor longitudinally along axis 400 from a first position imaging a first tissue group to a second longitudinal position to image tissue at a different depth along the axis. Separately or in combination with axially translation, the angulation of the distal portion 230 may be changed from alignment with axis 400 to alignment with axis 400′. The resulting change in angulation will allow the system to image aortic tissue at a different position in the anatomy. Finally, the forward looking sensor may be rotated about longitudinal axis 400 to change the orientation of the truncated image cone. For example, referring to FIG. 4b, a first truncated image cone containing field of view 402′ may be changed to a second truncated image cone containing field of view 402″′ by rotating the forward looking sensor 90° about the longitudinal axis 400.

Referring now to FIGS. 4a and 4b, there is shown a partial cross sectional view of an aorta 5 with aortic valve 14 and branching coronary arteries 16, 18 and saphenous vein graft 22. FIG. 4a includes an artificial valve delivery system 500 having a delivery catheter 510 and a prosthetic valve 520 in an undeployed condition. FIG. 4b illustrates the fully deployed prosthetic aortic valve 520 positioned across the nature aortic valve 14. During use, the distal portion 230 of the imaging system is positioned in the right atrium with the distal tip pointed at the aortic valve 14. From the stylized field of view 402 of FIG. 4a, the user can visualize valve leaflets 1 and 2 along with the diameter 532 of the annulus. Information concerning the health of the leaflets along with the diameter 532 of the aortic valve annulus can assist in selecting the proper size prosthetic valve 520. In addition, as shown in FIG. 4b, the distal portion 230 may be moved to align with axis 400′ to thereby create a field of view 402″. From field of view 402″, the user can determine the ascending aorta diameter 536 which may also assist in selecting the appropriate sized prosthetic valve. From the field of view 402″′, the user can estimate the appropriate frame height that will fit within the available space in the vessel or the distance of the coronary ostium to the aortic annulus. At least one advantage of using the forward looking imaging system is that multiple radiation exposures and contrast injections are not required to obtain information about the patient health and anatomy. Further, the user may also redirect the distal tip to examine the heart for ventricular thrombus or other indications which might exclude the patient from having the valve replacement procedure.

The width of the sinus of valsalva, 534, can be obtained as well as the distance 538. The image created by the forward looking image can provide valuable information with respect to the health and condition of the aortic valve and annulus. Specifically, as one non-limiting example, the calcification levels and stenosis severity can be assessed.

The imaging system can be utilized with additional image processing software to stitch together consecutive imaging planes to create a 3D image. To accomplish this in vivo, the transducer assembly is slowly deflected in a controlled fashion and in synch with the cardiac cycle to obtain multiple images from essentially the same location but at different orientations. These images are then electronically stitched together to form a composite image. The same effect can be achieved by rotating the catheter slowly by 180 degrees and in synch with the cardiac cycle without gyrating the catheter distal tip as it is rotated.

Once measurements and evaluations have been made using the forward looking ultrasound sensor, the physician may proceed with the valve placement procedure. Since the forward looking ultrasound device is positioned in the right atrium, it may remain in place during the valve placement procedure and can be used to provide visualization of the remaining steps of the procedure. Specifically, the physician must first pass guidewire 530 across the natural aortic valve 14. Images from the imaging system 200 can assist in positioning the guidewire at the proper valve crossing point. Once the guidewire is positioned across the aortic valve, the prosthetic valve 520 is delivered to the aortic valve. As shown in FIG. 4a, the valve 520 is “roughly” positioned across the aortic valve without expansion. The image 402 from the forward looking imaging device 200 is used to evaluate the position of the valve 520 relative to the patient anatomy. For example, the valve 520 shown in FIG. 4a extends too far below the aortic valve annulus and should be withdrawn slightly from the heart before expansion. As previously described above, the tip 220 may be positioned in alternative locations to thereby image different portions of the valve 520 and surrounding anatomy before final deployment. As an additional feature, the rough placement and adjustments before full deployment may be made during beating heart cycles and do not require rapid pacing of the heart which can be deleterious to elderly patients.

Once the valve 520 is determined to be in the proper location, the valve is deployed to anchor its position across the aortic valve. Referring to FIG. 4b, the Forward Looking ICE system may now be used to evaluate the placement of the fully deployed valve 520. Specifically, the physician can initially assess whether the valve was deployed in the desired position along the aorta. One aspect that can be confirmed is that the inferior portion of the valve is seated in the natural aortic valve annulus and does not extend too far into the heart. In another aspect of the method, the physician looks for blood flow into the coronary arteries 16 and 18 to confirm sufficient flow. The Forward Looking ICE system may detect the Doppler shift as blood moves toward or away from the sensor during each heart beat. The Forward Looking ICE system may also be used to evaluate the seal created between the valve annulus and the artificial valve. In a similar manner, the physician may image the superior portion of the valve 520 to assess whether the anchoring portion is fully seated against the aortic wall. All of the information gathered during the imaging process may be saved to the patient's medical record for later review and evaluation should revision surgery be needed.

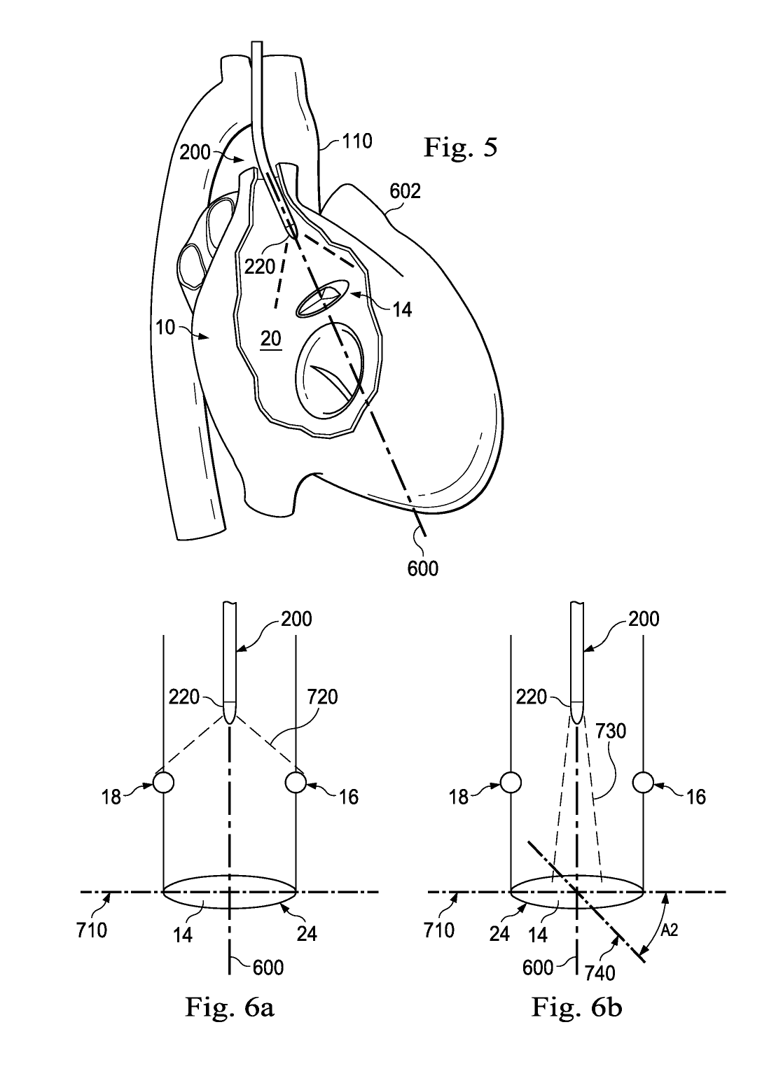

In an alternative approach, the Forward Looking ICE imaging system 200 may be inserted into the patient through the subclavian vein and positioned in the superior vena cava 20 as shown in FIG. 5. From this position, the Forward Looking ICE imaging system 200 may be maneuvered to image the aortic valve 14 from the superior vena cava. As shown in FIG. 5, the distal portion of the imaging system is aligned with axis 600 that extends from the superior vena cava 20 through the aortic valve 14. A portion of the right atrium 10 and ascending aorta is made transparent in FIG. 5 so the aortic valve and imaging system can be illustrated. The Forward Looking ICE imaging system 200 generates a truncated field of view 602 orient along axis 600. As discussed above, the field of view 602 may adjusted by moving or rotating the distal tip 220 along the axis 600 or by redirecting the distal tip to a new axis (not shown) to visualize tissue disposed a greater distance off of existing axis 600.

In still a further method of aortic valve placement, a Forward Looking ICE imaging device is advanced through the aortic arch 112 to visualize the aortic valve directly from the aorta. The Forward Looking ICE device may be advanced along the longitudinal axis to evaluate tissues at different depths within the body. In addition, as described in more detail above, once the aortic valve is within a field of view, the distal tip may be rotated to obtain alternative images for measurement and evaluation.

Referring now to FIGS. 6a and 6b, the Forward Looking ICE system 200 distal tip 220 is positioned in a first position along axis 600 to define a first field of view 720 extending along a first imaging plane. In the first imaging plane 710 shown in FIG. 6a, the system images the coronary ostiums leading to the coronary arteries 16 and 18 as well as providing an image of a portion of the aortic valve 14. It will be appreciated that the system transmits the ultrasound beam through the wall of the superior vena cava and the wall of the aorta to create the images. From the field of view 720 shown in the FIG. 6a, the user can determine the location of the coronary ostiums and take a first measurement across the aortic valve 14 to determine at least one diameter dimension of the aortic annulus in a first plane.

The imaging catheter can also be used to precisely deliver the 0.035 inch guidewire across the aortic valve. Patients undergoing TAVI procedures often have leaflets that do not fully open or are no longer opening symmetrically. As a result, it is sometimes difficult to deliver the 0.035 inch guidewire across the valve. Continual attempts to cross with the guidewire may chip off calcium that can lead to stroke or perforation of the aorta. By combining the forward looking modality and the 0.035 inch guidewire lumen the physician can visualize the guidewire as it is pushed across the valve.

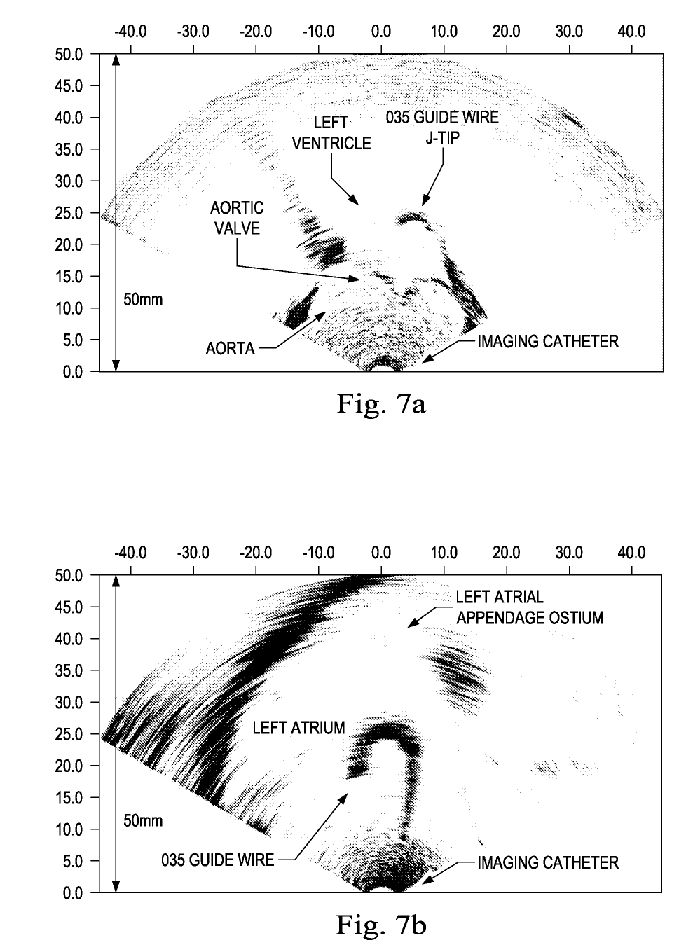

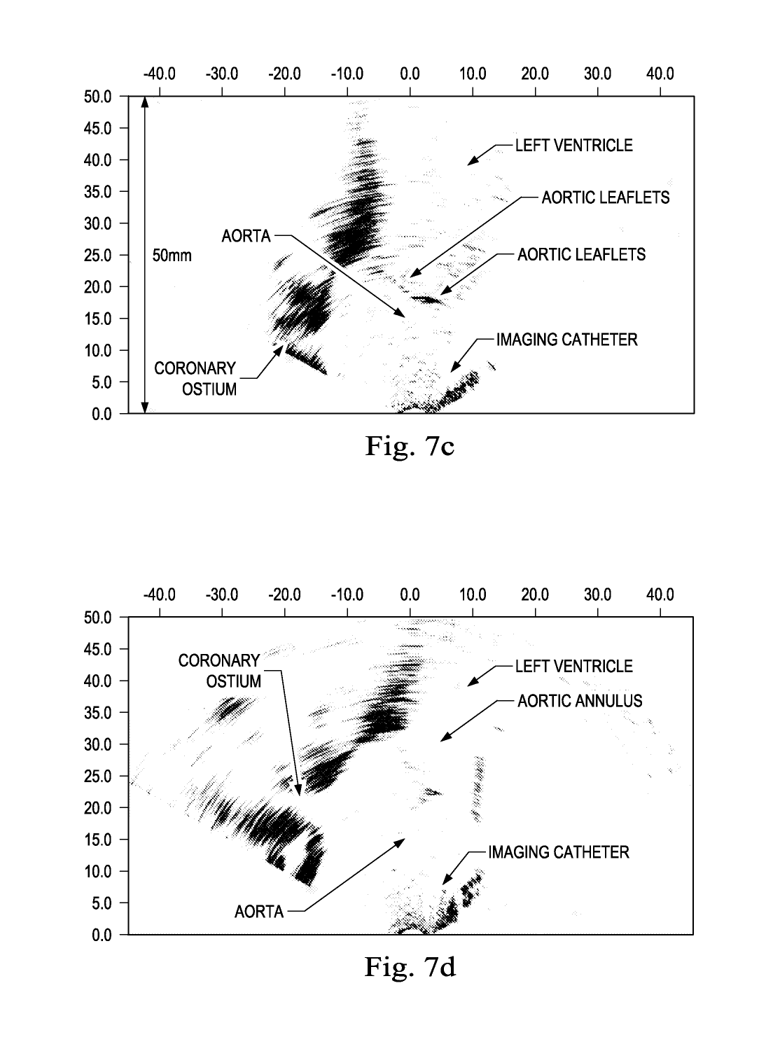

FIGS. 7a-7d illustrate images generated by a forward looking imaging catheter utilized in vivo during a porcine animal trial. Referring now to FIG. 7a, there is shown an image generated by the forward looking catheter of a 0.035 inch guidewire after it crosses the aortic valve. The forward looking imaging catheter is positioned in the aorta. As an additional illustration, FIG. 7b shows an image generated by the forward looking imaging catheter of a 0.035 inch guidewire being imaged in front of the Left Atrium Ostium. FIGS. 7c and 7d illustrate images generated by the forward looking imaging catheter positioned in the aorta. As shown, the distance to the coronary ostium can be precisely measured with the forward looking imaging catheter positioned in the aortic position.

Referring now to FIG. 6b, an alternative view of the aortic valve is generated from the Forward Looking ICE system 200 positioned in the superior vena cava. In this alternative view, the distal tip 220 has been rotated about the longitudinal axis 600. This new rotational position creates a new field of view 730 extending along offset image plane 740. In the illustrated embodiment, offset image plane 740 is offset from image plane 710 by angle A2. In one aspect, angle A2 is substantially 90°. Although in this new angular position the coronary ostiums are not visible, the user may take a second measurement of the aortic annulus 24 to determine a second diameter dimension of the aortic annulus in a second plane. It will be understood that the steps of rotating the distal tip 220 about the axis 600, in combination with perpendicular deflection, may be repeated as many times as desired to image and measure further features of the aorta and aortic valve. Once the physician has made sufficient measurements, an appropriately sized implant may be selected based on these measurements.

In one alternative technique, the Forward Looking ICE imaging device 200 is advanced to the superior vena cava. The Forward Looking ICE device 200 is then oriented as described above with respect to FIG. 3 to image the aortic valve from a lateral view. Measurements of the aortic valve are taken from the lateral view as described above with respect to FIGS. 4a and 4b. These measurements can be combined with the axial measurements obtained from the superior vena cava position to determine the appropriate size and positioning of the implant. In a further alternative, the lateral measurements are taken first and then the Forward Looking ICE imaging device is withdrawn into the position in the superior vena cava.

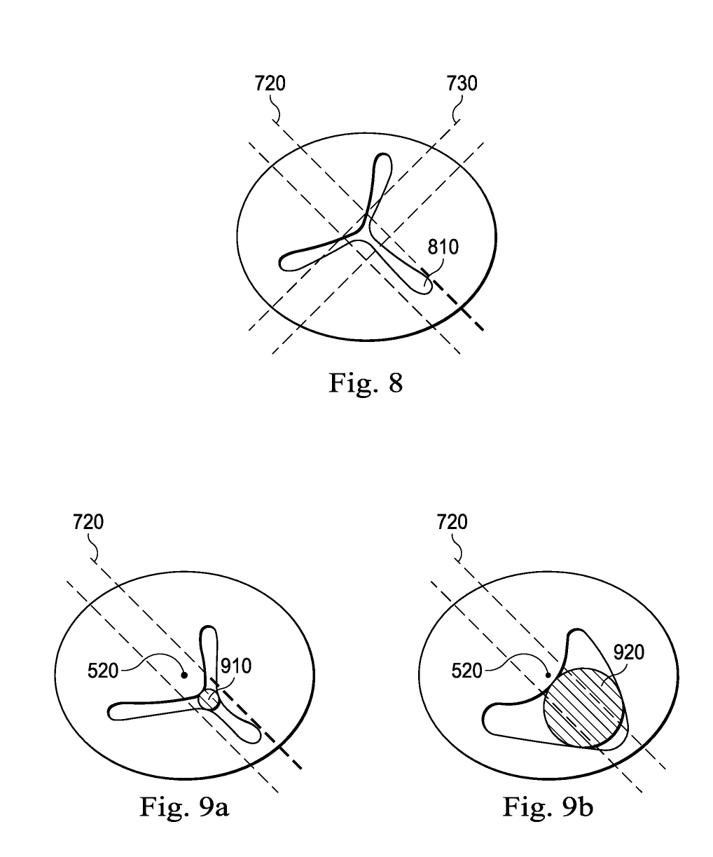

The method of implantation continues by providing a valve delivery system 500 over a guidewire 530 as described with respect to FIG. 4a. As previously explained, one difficulty in the procedure is passing the guidewire 520 through the aortic valve. With the generally superior to inferior view provided by the Forward Looking ICE imaging system 200 positioned in the aorta, the physician may use active Forward Looking ICE imaging to assist in passing the guidewire through the aortic valve. The aortic valve is initially imaged in a first orientation about axis 600 along image plane 710 to generate the first field of view 720 shown in FIG. 8. From this view, a surgeon can identify an area 810 that provides the easiest crossing location. In one aspect, the system automatically identifies through Doppler flow the area of greatest blood flow and suggests a crossing point to the surgeon. As cross reference, the surgeon may rotate the tip 220 to the second position to define field of view 730 and again evaluate the best location for crossing the aortic valve. The best field of view for crossing the aortic valve is then determined and the tip 220 is rotated to the appropriate position to generate the best field of view. Referring now to FIGS. 9a and 9b, the surgeon advances the guidewire 530 until it is visible in the field of view. FIG. 9a shows the guidewire 530 associated with the aortic valve leaflets in a generally closed positioned. The area 910 represents the ideal aiming area for passing the guidewire through the valve. FIG. 9b shows the guidewire associated with the aortic valve leaflets in a generally open position. The area 920 represents the area available for passing the guidewire 530 while the leaflets are in the open position. Once the appropriate field of view is displayed, the surgeon maneuvers the guidewire 530 to overlap with the area 910 or 920. With the guidewire 520 properly aligned, the surgeon then advances the guidewire through the aortic valve. The choice of whether to advance during the open valve condition or the closed valve condition may depend upon the size of the available area and the physician's ability to properly align the guidewire with the target location.

In an alternative embodiment, the imaging system has a guidewire targeting mode. In this mode, the physician images the aortic valve from one or more angular orientations. From this information, the system determines the best location for crossing the aortic valve. The system then prompts the user to rotate the distal tip to the desired field of view. Once in the appropriate field of view, an indicator is activated (such as a change in screen color to green, for example) to indicate to the user that the imaging probe is properly oriented. Once the correct field of view is displayed, the system then requests that the user advance the guidewire into the field of view. In this operating mode, the system will detect the strong echoes from the guidewire and direct the user to position the guidewire in the best valve crossing location. Once the field of view indicates that the guidewire is aligned with the best crossing location, the user may advance the guidewire to cross the aortic valve.

With the Forward Looking ICE imaging device positioned in the right atrium, the replacement valve 520 is advanced over the guidewire 530. Once the valve 520 is determined to be in the proper location by visualization with the Forward Looking ICE system, the valve is deployed to anchor its position across the aortic valve. From its position in the right atrium, the Forward Looking ICE system may now be used to evaluate the placement of the fully deployed valve 520. From the superior vena cava position, the physician can initially look for blood flow into the coronary arteries 16 and 18 to confirm that the valve placement did not block sufficient blood flow. The Forward Looking ICE system may detect the Doppler shift as blood moves toward or away from the sensor during each heart beat. The Forward Looking ICE system may also be used to evaluate the seal created between the valve annulus and the artificial valve. Utilizing Doppler flow, the Forward Looking ICE system may look for jets of blood flow passing between the exterior of the artificial valve 520 and an aortic valve annulus. The physician may also image the superior portion of the valve 520 to assess whether the anchoring portion is fully seated against the aortic wall. If leakage or misplacement is detected, the valve may be further manipulated to correct the placement error or removed completely if necessary. All of the information gathered during the imaging process may be saved to the patient's medical record for later review and evaluation should revision surgery be needed.

As described with other embodiments above, the Forward Looking ICE imaging device may also be utilized after valve placement to verify position and sealing.

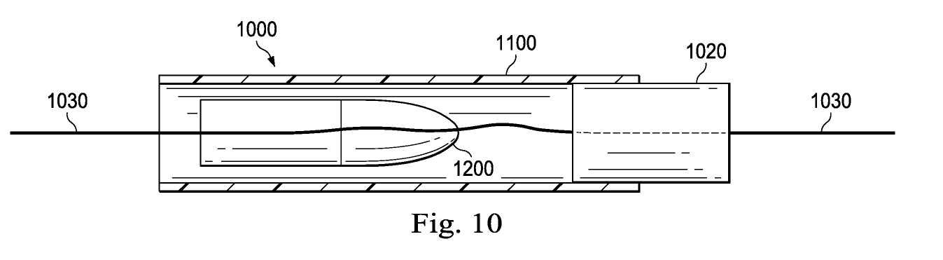

Referring now to FIG. 10, there is illustrated a valve delivery system 1000 incorporating a Forward Looking ICE imaging system 1200. As described above, the delivery system is advanced over a guidewire 1030 previously positioned across the aortic valve. The valve 1020 is positioned across the aortic valve and deployed to maintain its position. The Forward Looking ICE imaging device 1200 is then advanced from delivery catheter 1100 and used to evaluate valve positioning and sealing against the native aortic annulus using color doppler.

In one aspect, the combination system of FIG. 10 is used in combination with a Forward Looking ICE device positioned in the right atrium as described above with respect to FIG. 3. The right atrium Forward Looking ICE device may be used alternatively or simultaneously with the aortic Forward Looking ICE device to more accurately evaluate the natural anatomy and verify proper placement of the aortic valve.

In still a further aspect, more than one Forward Looking ICE imaging device may be deployed within the patient simultaneously. More specifically, a physician may position a Forward Looking ICE imaging device in the right atrium consistent with FIG. 3 to obtain aortic valve information from a generally lateral view. With the Forward Looking ICE device residing in the right atrium, a second Forward Looking ICE imaging device may be positioned in the aorta consistent with FIG. 6 or in the superior vena cava to obtain aortic valve information from a generally axial view. In one feature, the display system simultaneously shows the imaging information from the lateral view and from the axial view. This information may then be used determine the proper size of the valve, assist with rough placement of the valve during normal beating heart cycles and finally to assess valve placement and function after deployment. In one aspect, the above described steps are performed without angiography and the associated contrast media.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A medical method, comprising:
positioning a guidewire and a forward looking imaging device in the vasculature of a patient;
advancing the guidewire and forward looking imaging device to a position adjacent the aortic valve;
imaging the aortic valve with the forward looking imaging device to obtain a valve image and determine properties of the valve;
crossing the aortic valve with at least a distal portion of the guidewire utilizing the valve image.

2. The method of claim 1, wherein said imaging includes imaging tissue positioned distally beyond a distal tip of the guidewire.

3. The method of claim 1, wherein the valve image is an axial image of the valve.

4. The method of claim 1, wherein said advancing includes advancing along a first path, the method further including determining a second path based on the valve image and aligning a least a distal tip of the guidewire with the second path and said crossing includes advancing the distal tip along the second path.

5. The method of claim 4, wherein the first path defines a first longitudinal axis for at least the distal tip and said aligning includes rotating or moving the distal tip laterally away from the first longitudinal axis.

6. The method of claim 1, wherein the advancing includes positioning the forward looking imaging device in the superior vena cava oriented to view the aortic valve.

7. The method of claim 1, wherein the advancing includes positioning the forward looking imaging device in the right atrium oriented to view the aortic valve.

8. The method of claim 1, wherein the advancing includes positioning the forward looking imaging device within the aorta oriented to view the aortic valve.

9. The method of claim 1, wherein the properties include a determination of the distance from the coronary ostia.

10. The method of claim 1, wherein the properties include a determination of the annulus shape and diameter.

11. The method of claim 1, wherein the properties include an evaluation of calcium deposits on the aortic valve.

12. The method of claim 1, wherein the imaging includes developing three dimensional properties of the aortic valve.

13. The method of claim 1, further including advancing to a second location to generate a second set of images of the aortic valve and viewing a comparison of the first set of images from the first location to the second set of images from the second location.

14. The method of claim 13, further including synchronizing the images viewed with a certain portion of the heartbeat.

15. The method of claim 13, further including utilizing the forward looking imaging device to evaluate blood flow adjacent the aortic valve.

16. The method of claim 15, wherein said evaluating includes evaluation of regurgitation of blood through the aortic valve.

17. The method of claim 15, wherein said evaluating includes evaluation of the blood flow through the coronary ostium.

18. The method of claim 15, wherein said utilizing includes visualizing a colorized image to evaluate blood flow.

19. The method of claim 1, wherein the imaging device has a distal end defining an imaging plane, further including deflecting the distal end in a first direction to locate the valve major axis and deflecting the image plane in a second direction to locate the valve minor axis.

20. A medical method, comprising:
positioning a forward looking imaging device in the vasculature of a patient;
advancing the forward looking imaging device into the aorta adjacent the natural aortic valve;
imaging the natural aortic valve with the forward looking imaging device to obtain a valve image and determine properties of the natural aortic valve;
placing an unexpanded replacement valve in the annulus of the natural aortic valve utilizing the valve image; and
imaging the position of the unexpanded replacement valve.

21. The method of claim 20, further including repositioning the unexpanded replacement valve within the natural aortic valve annulus in response to imaging the position of the unexpanded replacement valve.

22. The method of claim 20, further including visualizing the deployment of the replacement valve within the natural aortic valve annulus and evaluating the placement of the replacement valve.

23. The method of claim 22, wherein said evaluating includes viewing an image of blood flow through the replacement valve to consider leakage between the replacement valve and the aortic valve annulus.

24. The method of claim 23, wherein the image of blood flow through the replacement valve includes blood flow during systole to allow visualization of blood flow in the wrong direction.

25. The method of claim 22, wherein the method further includes utilizing the imaging device to take hemodynamic measurements following deployment of the replacement valve.

26. A system, comprising:
a processor configured to receive imaging signals from an aortic imaging device;
a visual display;
a delivery catheter including:
a prosthetic valve positioned adjacent a distal end; and
a forward looking imaging device positioned adjacent said prosthetic valve, wherein said imaging device is positioned to image tissue extending distally beyond said valve and generates image signals; and
a connection between said forward looking imaging device and said processor, said connection providing said image signals to said processor.

27. The system of claim 26, wherein said imaging device is placed proximal of the prosthetic valve along the delivery catheter.

28. The system of claim 26, wherein said processor is configured to display an image of the aortic valve with an indication of the best pathway through the valve and an indication of current image device alignment in relation to the best pathway.

29. The system of claim 26, further including a second forward imaging device sized for placement in the right atrium, said second imaging device generating second image signals, said processor receiving said second image signals and configured to generate a second image based on said second signals.

30. The system of claim 29, wherein said processor is further configured to generate a three dimensional image of at least a portion of the aortic valve based on input from said first and second image signals.
